(19) United States
(12) Patent Application Publication
(10) Pub. No.: US 2008/0004656 A1
(43) Pub. Date: Jan. 3, 2008

(54) SURGICAL INSTRUMENT WITH DETACHABLE TOOL ASSEMBLY

(75) Inventor: Steve Livneh, Amherstburg (CA)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(73) Assignee: BOVIE MEDICAL CORPORATION, St. Petersburg, FL (US)

(21) Appl. No.: 11/772,517

(22) Filed: Jul. 2, 2007

Related U.S. Application Data
(63) Continuation-in-part of application No. 11/380,797, filed on Apr. 28, 2006.
(60) Provisional application No. 60/818,159, filed on Jun. 30, 2006.

(51) Int. Cl. A61B 17/28

(52) U.S. Cl. 606/205

(57) ABSTRACT

A surgical instrument includes a detachable tool assembly. The assembly includes a connection rod and a tool, such as a pair of jaws or an electrode. The instrument also includes a shaft operatively connected to a handle movable between operating positions and an ejected position. A tubular housing encloses the shaft and a collet. The collet is attached to the rod and movable between the positions. The fingers of the collet are flared apart in the ejected position but collapse to capture the connection rod in the operating positions. A tongue-and-groove arrangement provides a secondary coupling between the assembly and the housing when the handle is in the operating positions.

### CROSS REFERENCE TO RELATED APPLICATION

The subject patent application claims priority to and all the benefits of U.S. Provisional Patent Application Ser. No. 60/818,159 which was filed on Jun. 30, 2006, the entire specification of which is expressly incorporated herein by reference.

### BACKGROUND OF THE INVENTION

1. Field of the Invention

The invention relates to a surgical instrument having a detachable tool assembly.

2. Description of the Related Art

Surgical instruments, such as forceps, are well known in the art for use in surgery and other medical procedures. Commonly, forceps, such as Kelly forceps, have a two piece construction like scissors, but are utilized to grip instead of cut. These common forceps includes a pair of handles attached to a pair of jaws.

During advanced endoscopic and arthroscopic surgeries, a surgeon may require several different styles and sizes of jaws depending on a wide variety of factors. To save on disposal and sterilization costs, medical device suppliers began offering surgical instruments with detachable tool assemblies.

One such instrument is disclosed in U.S. Pat. No. 5,893,875 (the '875 patent) to O'Connor et al. The instrument of the '875 patent includes a handle connected to a shaft. The instrument also includes a detachable tool;, such as scissors. A first sleeve connected to the tool mates with a second sleeve connected to the shaft. Disconnection of the tool from the shaft may be clumsy and requires two-handed rotation of components.

Therefore, there remains an opportunity for a surgical instrument with an easily detachable tool assembly that can be operated with a single hand.

### SUMMARY OF THE INVENTION AND ADVANTAGES

The present invention provides a surgical instrument. The instrument includes a shaft having a proximal end and a distal end. A housing surrounds at least a portion of the shaft. At least one handle is operatively connected to the proximal end of the shaft for longitudinally actuating the shaft within the housing. The instrument also includes a detachable tool assembly having a connection rod having a proximal end and a distal end. At least one tool is operatively connected to the connection rod. The instrument further includes a collet disposed within the housing and detachably coupling the reciprocating and the connection rod.

The surgical instrument of the present invention allows for quick detachment of the detachable tool assembly merely by actuating the handle. This detachment can be formed by an operator of the instrument (e.g., a surgeon or assistant) with a single hand, thus allowing the operator's other hand to be free to perform other tasks. Furthermore, the operator need not remove his hand from the handles of the instrument to detach the tool assembly. This prevents potential contamination by handling of the tool assembly.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 is a perspective view of a first embodiment of a surgical instrument with a detachable tool assembly having a pair of jaws and operable by a pair of handles;

FIG. 2 is a cross-sectional view of the first embodiment of the surgical instrument with the jaws and handles in an open position;

FIG. 3 is a cross-sectional view of the first embodiment of the surgical instrument with the jaws and handles in a closed position;

FIG. 4 is a cross-sectional view of the first embodiment of the surgical instrument with the jaws and handles in an ejected position;

FIG. 5 is a perspective view of the detachable tool assembly of the first embodiment detached from a housing;

FIG. 6 is a cross-sectional view of the jaws of the detachable tool assembly of the first embodiment detached from the housing;

FIG. 7 is an exploded view of a portion of the first embodiment of the surgical instrument showing the detachable tool assembly, a clevis component, the housing, and a shaft;

FIG. 8 is a cross-sectional view of the jaws of the detachable tool assembly of the first embodiment in the closed position;

FIG. 9 is a perspective view of a collet with fingers in a compressed position;

FIG. 10 is a perspective view of the collet with fingers in a flared position;

FIG. 11 is a perspective view of a first configuration of a coupling between the collet and the housing;

FIG. 12 is a cross-sectional view of the housing in the first configuration;

FIG. 13 is a perspective view of the detachable tool assembly of the first embodiment in a second configuration;

FIG. 14 is a cross-sectional view of the detachable tool assembly connected to the housing in the first embodiment and the second configuration showing the jaws in the open position;

FIG. 15 is a cross-sectional view of the detachable tool assembly connected to the housing in the first embodiment and the second configuration showing the jaws in the closed position;

FIG. 16 is a cross-sectional view of the detachable tool assembly detached from the housing in the first embodiment and the second configuration;

FIG. 17 is a perspective view of the second configuration of the coupling between the collet and the housing;

FIG. 18 is a cross-sectional view of the housing in the second configuration;

FIG. 19 is a perspective view of the detachable tool assembly and housing of the first embodiment in a third configuration;

FIG. 20 is a cross-sectional view of the detachable tool assembly connected to the housing in the first embodiment and the third configuration showing the jaws in the open position;

FIG. 21 is a cross-sectional view of the detachable tool assembly connected to the housing in the first embodiment and the third configuration showing the jaws in the closed position;

FIG. 22 is a cross-sectional view of the detachable tool assembly detached from the housing in the first embodiment and the third configuration;

FIG. 23 is a perspective view of the third configuration of the coupling between the collet and the housing;

FIG. 24 is a cross-sectional view of the housing in the third configuration;

FIG. 25 is a perspective view of the detachable tool assembly and the housing in the first embodiment and in a fourth configuration;

FIG. 26 is a cross-sectional view of the detachable tool assembly connected to the housing in the first embodiment and the fourth configuration showing the jaws in the open position;

FIG. 27 is a cross-sectional view of the detachable tool assembly connected to the housing in the first embodiment and the fourth configuration showing the jaws in the closed position;

FIG. 28 is a cross-sectional view of the detachable tool assembly detached from the housing in the first embodiment and the fourth configuration;

FIG. 29 is a perspective view of the fourth configuration of the coupling between the collet and the housing;

FIG. 30 is a cross-sectional view of the housing in the fourth configuration;

FIG. 31 is a perspective view of a second embodiment of the surgical instrument showing the clevis component affixed to the housing;

FIG. 32 is a cross-sectional view of the second embodiment showing the detachable tool assembly connected to the housing and the jaws in the open position;

FIG. 33 is a cross-sectional view of the second embodiment showing the detachable tool assembly connected to the housing and the jaws in the closed position;

FIG. 34 is a cross-sectional view of the detachable tool assembly of the second embodiment showing a pin assembly;

FIG. 35 is a perspective view of the clevis component of the second embodiment;

FIG. 36 is an exploded perspective view of the detachable tool assembly of the second embodiment;

FIG. 37 is a cross-sectional view of a third embodiment of the surgical instrument showing the detachable tool assembly implemented as an electrode;

FIG. 38 is a perspective view of the electrode;

FIG. 39 is a cross-sectional view of the third embodiment in a first configuration showing the electrode connected to the housing;

FIG. 40 is a cross-sectional view of the third embodiment in the first configuration showing the electrode detached from the housing;

FIG. 41 is a cross-sectional view of the third embodiment in a second configuration showing the electrode detached from the housing;

FIG. 42 is a cross-sectional view of the third embodiment in a third configuration showing the electrode detached from the housing;

FIG. 43 is a cross-sectional view of the third embodiment in a fourth configuration showing the electrode detached from the housing; and

FIG. 44 is a perspective view of a fourth embodiment of the surgical assembly showing the electrode detached from the housing.

### DETAILED DESCRIPTION OF THE INVENTION

Referring to the Figures, wherein like numerals indicate corresponding parts throughout the several views, a surgical instrument 50 is shown.

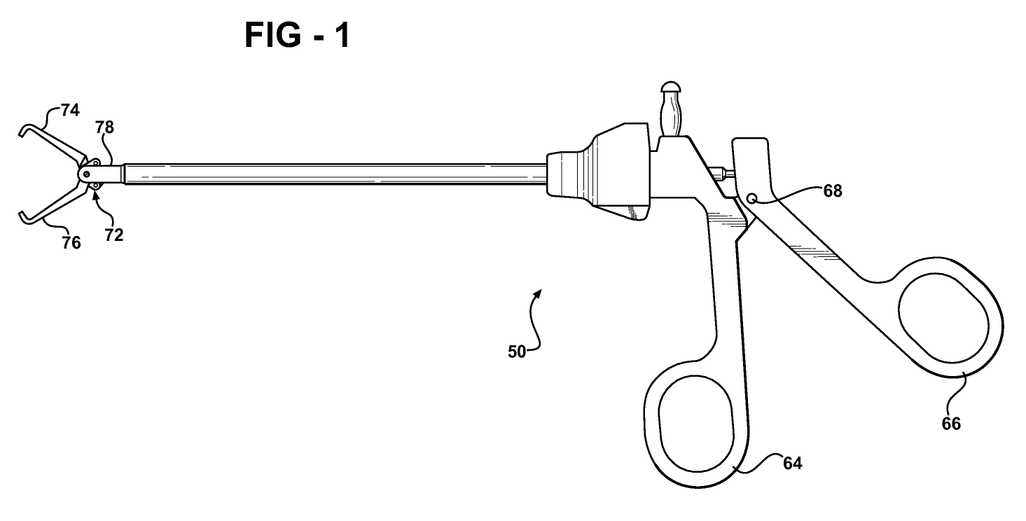

In a first embodiment, as shown in FIG. 1, the surgical instrument 50 is forceps primarily for use in performing endoscopic or arthroscopic surgery, specifically for the grasping of objects during such surgery. The components of the surgical instrument 50 are typically formed of a metal, such as a stainless steel. However, the various components of the surgical instrument may be formed of other materials as are known to those skilled in the art.

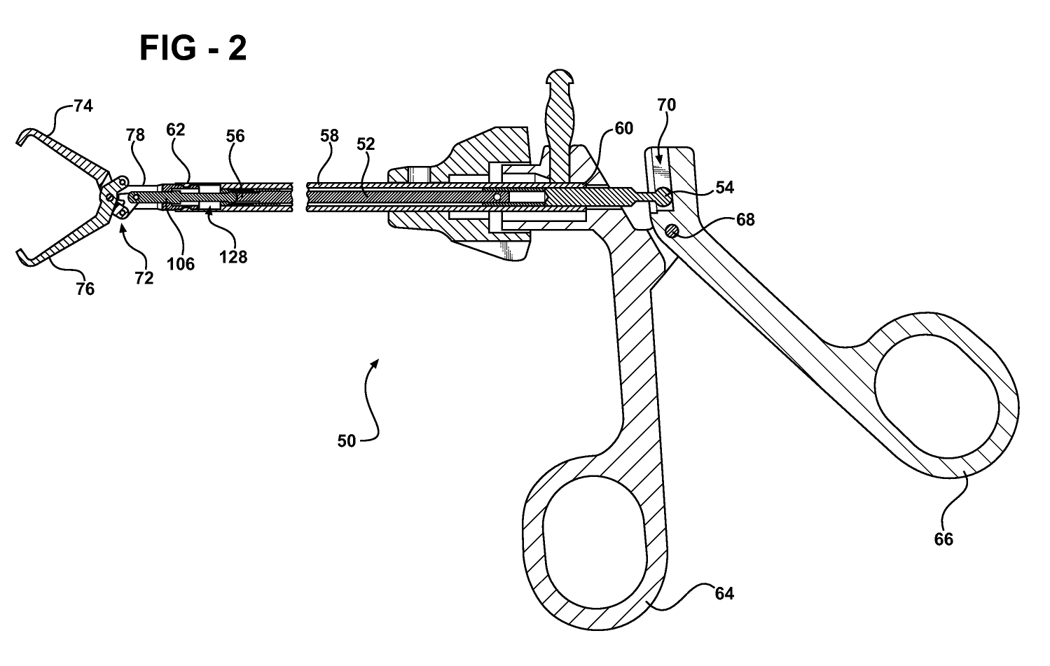

Referring now to FIG. 2, the surgical instrument 50 includes a shaft 52 having a proximal end 54 and a distal end 56. Preferably, the shaft 52 is rod-shaped with a generally circular cross section. However, the cross section of the shaft 52 may define other shapes. For instance, the cross section of the shaft 52 may be square or triangular (both not shown).

The shaft 52 is disposed at least partially in a housing 58. That is, the housing 58 surrounds at least a portion of the shaft 52. The housing 58 is preferably tubular-shaped and includes a proximal end 60 and a distal end 62. The housing preferably defines a circular cross section with a hollow center for accommodating the shaft 52. As with the shaft 52, the cross section of the housing 58 may also define other shapes.

At least one handle 64, 66 is operatively connected to the shaft 52, preferably at the proximal end 54 of the shaft 52. In the first embodiment, the surgical instrument 50 includes a fixed handle 64 and a movable handle 66. The fixed handle 64 is attached to the proximal end 60 of the housing 58 and does not move with respect to the housing 58. The movable handle 66 is pivotally connected to the fixed handle 64. A pin 68 disposed through holes (not labeled) in each of the handles 64, 66 provides this pivotable connection.

In the first embodiment, the movable handle 66 is connected to the proximal end 54 of the shaft 52. The proximal end 54 of the shaft 52 defines a ball shape. The movable handle 66 defines a groove 70 for accommodating this ball-shaped proximal end 54. As the movable handle 66 is actuated, the proximal end 54 slides in the groove 66 to longitudinally actuate the shaft 52 within the housing 58. Said another way, the shaft 52 reciprocates, i.e., moves back and forth, within the housing 58 when actuated by the movable handle 66. The movement of the movable handle 66 and the shaft 52 within the housing 58 can be seen at various positions in FIGS. 2-4 and described in more detail below.

The surgical instrument 50 includes a detachable tool assembly 72. The detachable tool assembly 72 is operatively connected to the shaft 52. The detachable tool assembly 72 includes at least one tool (not numbered). In the first embodiment, the tool is further defined as a first jaw 74 and a second jaw 76 for grasping an object. Those skilled in the art realize numerous design possibilities for the jaws 74, 76 other than that shown in the figures. The detachable tool assembly 72 is easily separable from the shaft 52 and the housing 58 to allow a user of the surgical instrument 50 to quickly change the desired tool. The jaws 74, 76 of the first embodiment are pivotally fastened together such that both jaws 74, 76 are movable as described in detail below. However, in alternative embodiments, one of the jaws 74, 76 could be fixed.

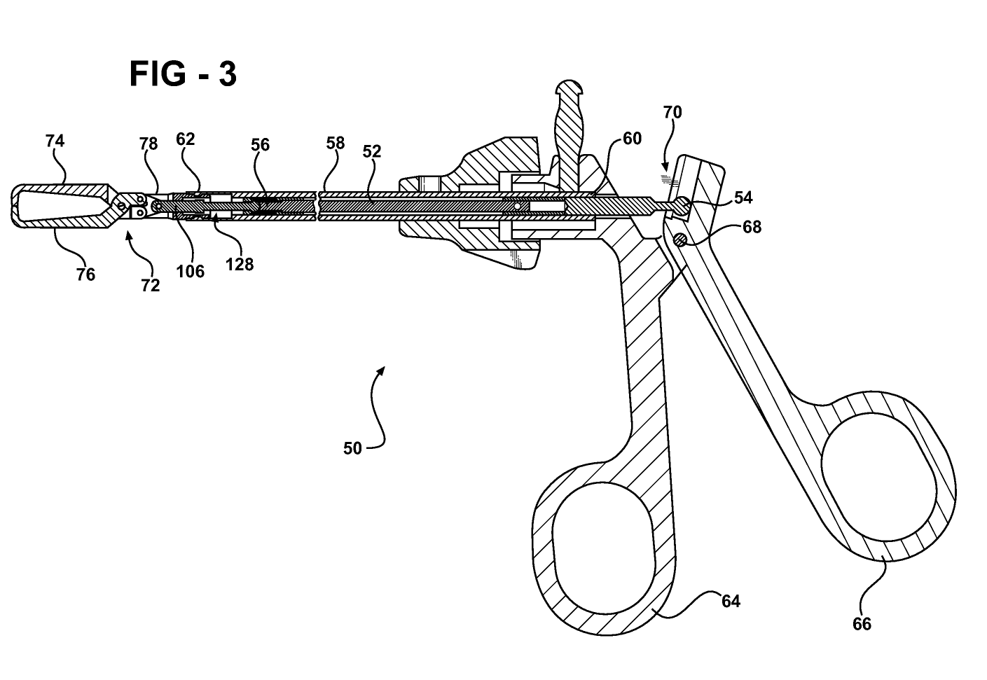

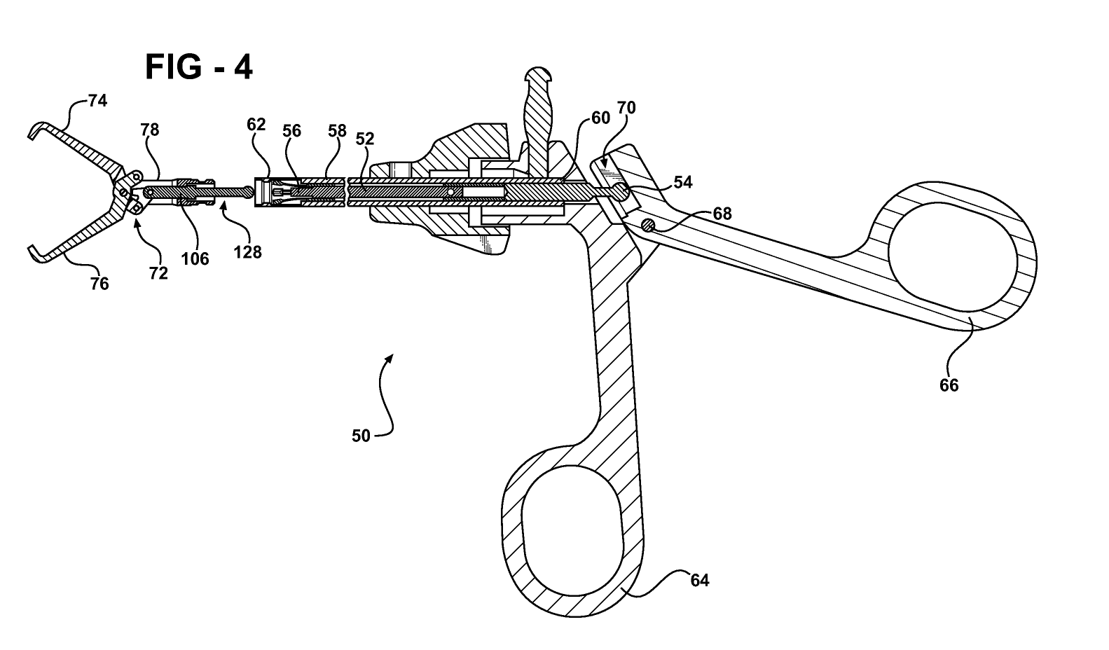

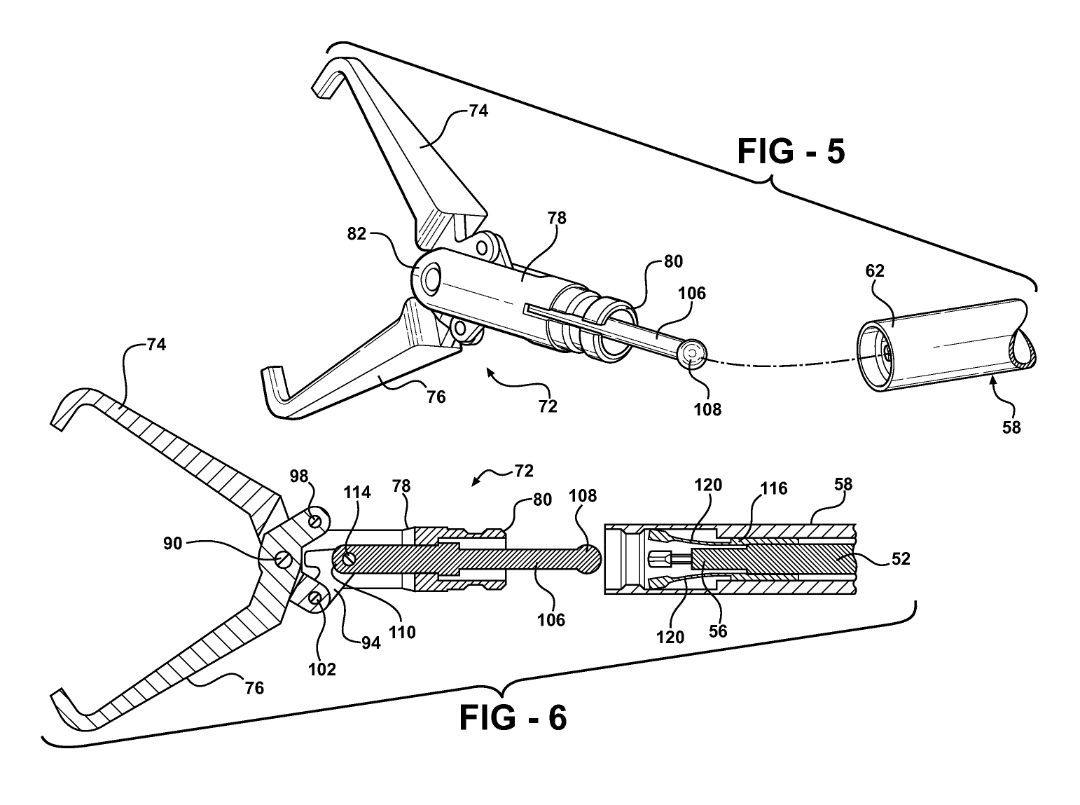

The movable handle 66, shaft 52, and jaws 74, 76 are movable in a plurality of operating positions. These positions include an open position as shown in FIG. 2, a closed position as shown in FIG. 3, as well as numerous positions in-between. The movable handle 66 and shaft 52 are also movable to an ejected position, where the detachable tool assembly 72 is detached from the instrument 50, as shown in FIG. 4. The assembly 72, detached from the housing 58, can also be seen in FIGS. 5 and 6.

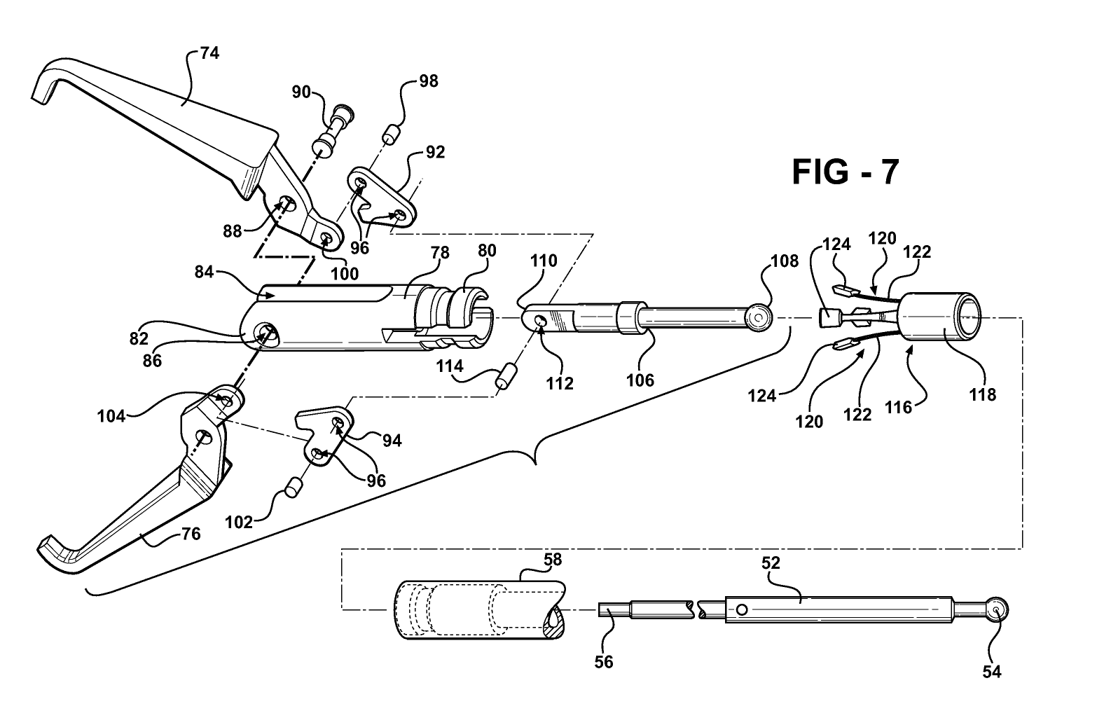

Referring to FIGS. 5-7, the assembly 72 includes a clevis component 78 having a proximal end 80 and a distal end 82. The proximal end 80 of the clevis component 78 is detachably coupled to the distal end 62 of the housing 58. To mate with the housing 58, the clevis component 78 preferably defines a circular cross section, but other shapes may alternatively be suitable. As best seen in FIG. 7, the clevis component 78 component defines a slot 84 extending inward from the distal end 82 to separate a pair of legs (not numbered). A clevis hole 86 is also defined through each leg of the clevis component 78, adjacent the distal end 82. The clevis holes 86 are generally perpendicular to the slot 84.

In the first embodiment, each jaw 74, 76 defines a pivot hole 88. A clevis pin 90 is disposed through the pivot holes 88 and the clevis holes 86 to facilitate pivotal movement of the jaws 74, 76 about the clevis pin 90.

The assembly 72 of the first embodiment also includes a first link 92 and a second link 94. Preferably, the links 92, 94 are at least partially disposed within the slot 84 of the clevis component 78. It is also preferred that two connection holes 96 are defined in each link 92, 94. The first link 92 is connected to the first jaw 74 via a first connection pin 98 disposed through one of the connection holes 96 in the first link 92 and a connection hole 100 in the first jaw 74. The second link 94 is connected to the second jaw 76 via a second connection pin 102 disposed through one of the connection holes 96 in the second link and a connection hole 104 in the second jaw 76.

The assembly 72 of the first embodiment also includes a connection rod 106 having a proximal end 108 and a distal end 110. The proximal end 108 preferably defines a ball shape. A hole 112 is defined adjacent to the distal end 110 of the connection rod 106. A pin 114 connects the hole 112 of the connection rod 108 to one of the connection holes 94 of each of the links 92, 94.

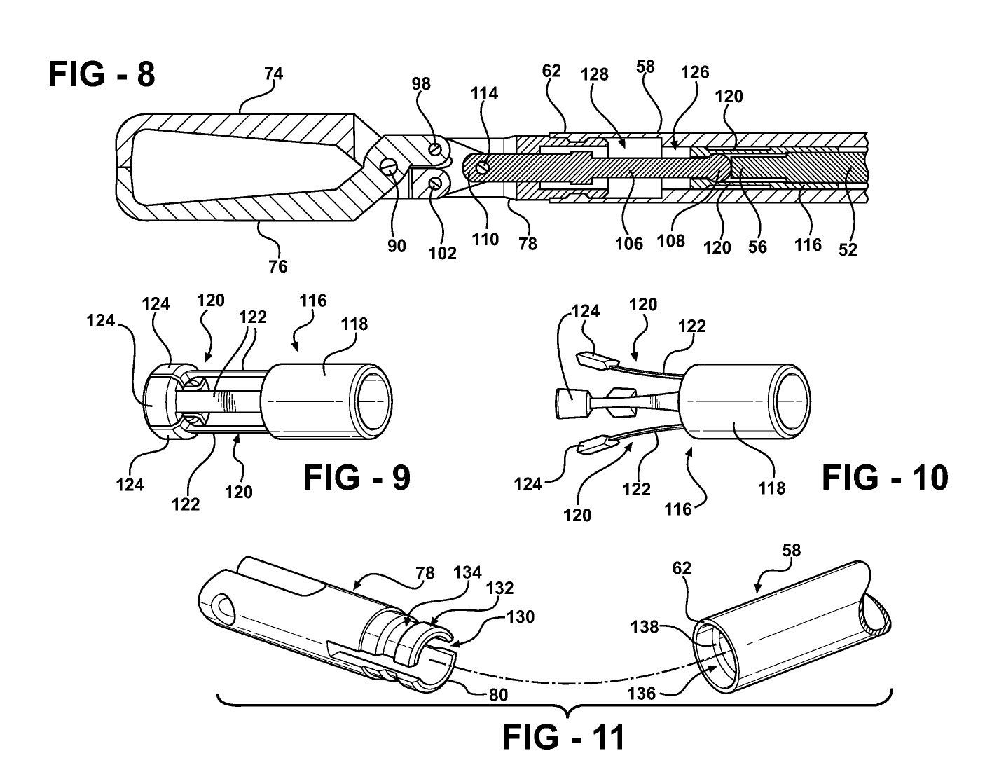

The interconnections described above allow the jaws 74, 76 of the assembly 72 to be movable in response to reciprocating movement of the connection rod 106. As the connection rod 106 is pulled away from the clevis component 78, the jaws 74, 76 move towards one another and into the closed positions, as is shown in FIGS. 3 and 8. As the connection rod 106 is pushed towards the clevis component 78, the jaws 74, 76 expand away from one another into the open position, as is seen in FIG. 2.

As shown in FIGS. 6-10, the surgical instrument 50 includes a collet 116 for detachably coupling the connection rod 106 to the shaft 52. The collet 116 is disposed within the housing 58. The collet 116 includes a base 118 and a plurality of expandable fingers 120 extending from the base 118. The base 118 of the collet 116 is attached to the distal end of the shaft 52. This attachment of the collet 116 to the shaft 52 may be accomplished by numerous techniques, including, but not limited to, welding, soldering, a fastener, or an adhesive. Those skilled in the art appreciate other techniques to attach the collet 116 to the shaft 52. Alternatively, the shaft 52 and collet 116 may be integrally formed together as a unitary component.

Referring now specifically to FIGS. 9 and 10, each finger 120 of the collet 116 includes a stem 122 extending from the base and a tip 124 disposed atop of the stem 122. Preferably, each stem 122 is a substantially flat and thin piece of metal. This allows the tips 124 of the fingers 120 to flare apart from one another when not compressed. However, when compressed, the tips 124 of the fingers 120 come together to form a hoop (not numbered). The ball-shaped proximal end 108 of the connection rod 106 is captured between the base 118 and the tips 124 of the fingers 120. Said another way, the collet 116 encompasses the ball-shaped proximal end 108. The connection rod 106, except for the ball-shaped proximal end 108, extends through a center (not numbered) of the hoop when the ball-shaped proximal end 108 is captured by the collet 116.

Referring to FIGS. 6 and 8, the housing 58 preferably defines a central opening 126 and a cavity 128. The cavity 128 is disposed adjacent the distal end 62 of the housing 58. The cavity 128 has a cross-sectional width greater than a cross-sectional width of the central opening 126. In the preferred embodiment, where the housing 58 has a circular cross section, the cavity 128 has a diameter that is greater than a diameter of the central opening 126.

Since the base of the collet 116 is attached to the distal end 56 of the shaft 52, the collet 116 moves within the housing as the shaft 52 reciprocates based on motion of the movable handle 66. The collet 116 is movable between the ejected position and the operating positions. In the operating positions, the fingers 120 of the collet 116 are substantially disposed within the central opening 126, such that the fingers 120 are collapsed to capture the ball-shaped proximal end 108 of the connection rod 106. In the ejected position, the fingers 120 of the collet 116 are substantially disposed within the cavity 128. As such, the fingers 120 flare away from one another in the cavity 128 to release the connection rod 106. Therefore, as the movable handle 66 is moved away from the fixed handle 64, the collet 116 moves into the ejected position, where the detachable tool assembly 72 can be removed from the surgical instrument 50. Even in the ejected position, the fingers 120 of the collet 116 are disposed within the housing. Thus, the fingers 120 are protected from accidental damage and breakage.

In addition to the coupling provided by the collet 116 and the connection rod 106, a secondary coupling is provided by the clevis component 78 and the housing 58. Specifically, the proximal end 80 of the clevis component 78 is removably coupled to the distal end 62 of the housing 58. As the movable handle 66 forces the shaft 62 in the ejected position, the shaft 62 forces the decoupling of the clevis component 78 from the housing 58. Thus, the surgical instrument 50 provides two separate couplings that are both controlled by the one-handed operation of the movable handle 66. This provides an extremely stable connection between the detachable tool assembly 72 and the housing 58.

The present invention discloses numerous configurations for achieving this secondary coupling. A first configuration of the secondary coupling is shown in FIGS. 5-8 and 11-12. Referring to FIG. 11, the clevis component 78 defines a pair of slits 130 protruding inward from the proximal end 80. The proximal end 80 of the clevis component 78 also defines a recessed outer surface 132 with a groove 134 indented into the recessed outer surface 132. The distal end 62 of the housing 58 defines an inner surface 136 with a tongue 138 protruding inward. The tongue 138 of the housing 58 and the groove 134 of the clevis component 78 mate to couple the housing 58 and clevis component 78 together. The slit 130 allows the proximal end 80 of the clevis component 78 to compress, thus allowing connection and disconnection of the clevis component 78 from the housing 58.

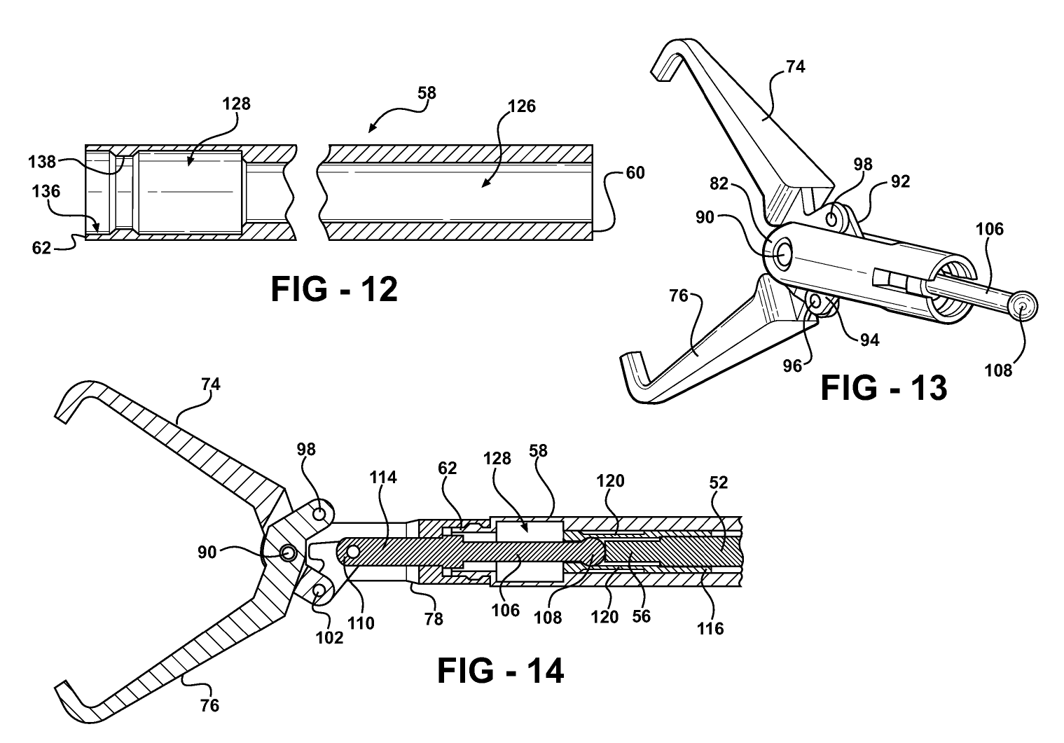

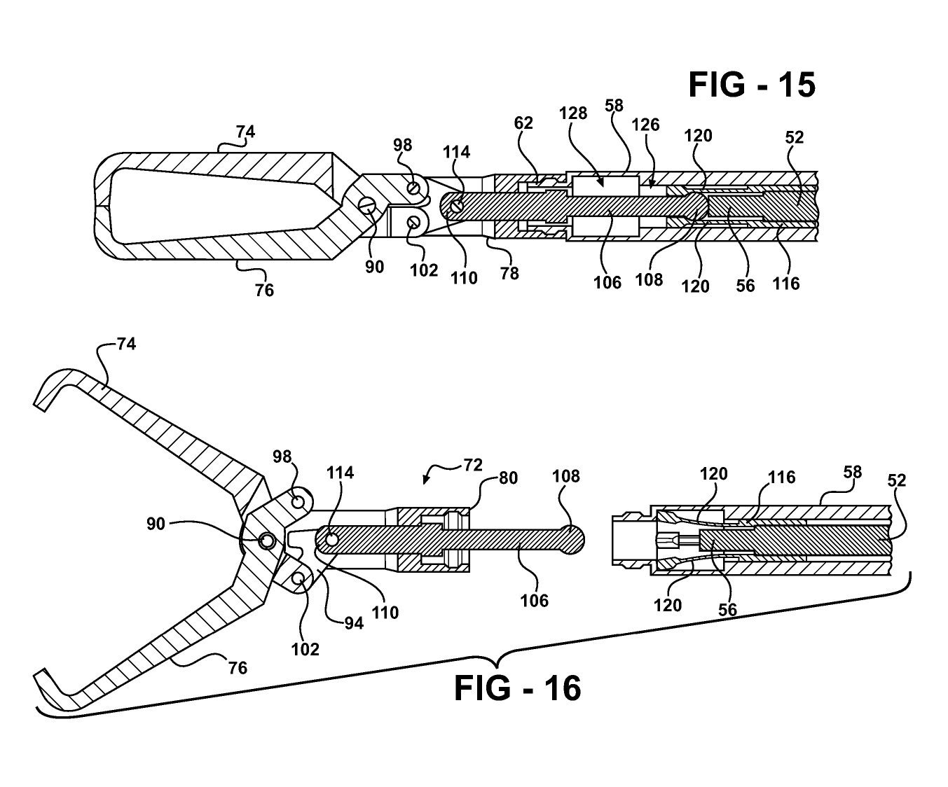

A second configuration of the secondary coupling is shown in FIGS. 13-18. Referring specifically to FIG. 17 the distal end 62 of the housing 58 defines a recessed outer surface 140 with a tongue 142 projecting outward from the outer surface 140. The clevis component 78 defines an inner surface 144 defining a groove 146 for mating with the tongue 142. The slit 130 protrudes inward from the proximal end 80 of the clevis component 78.

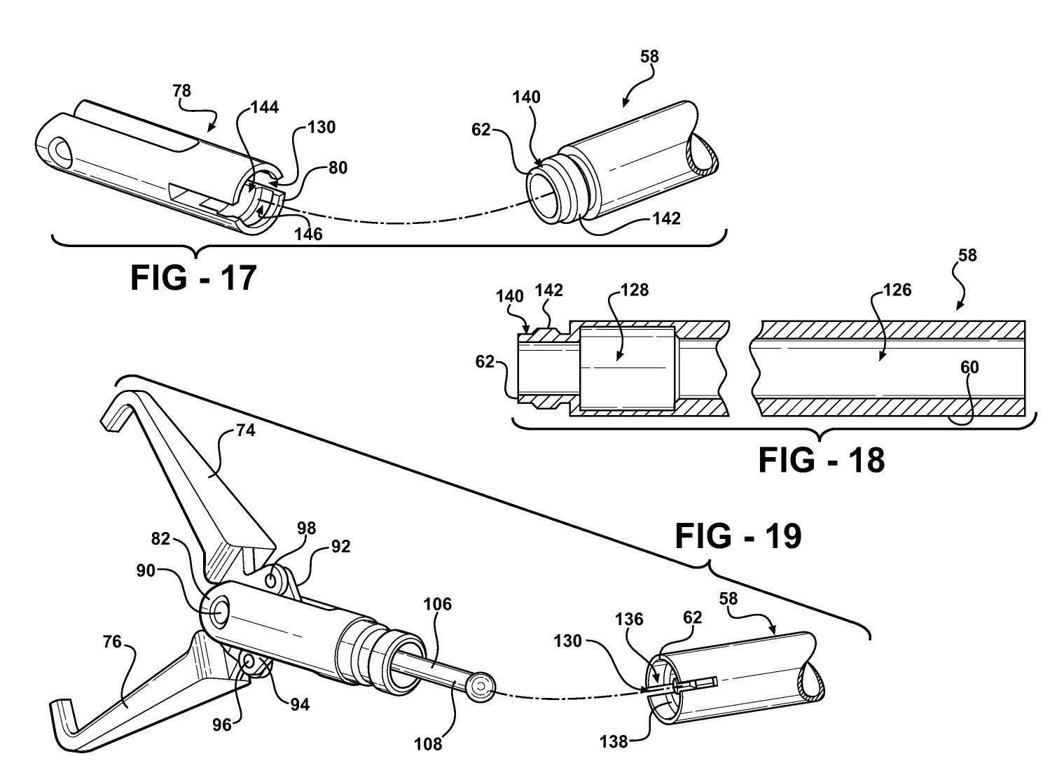

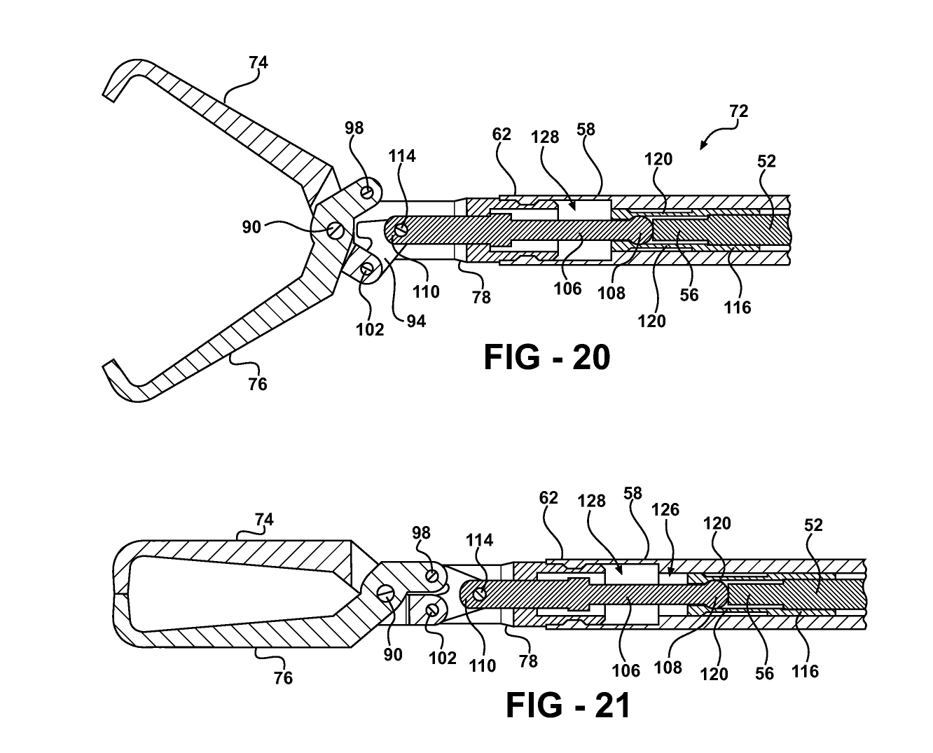

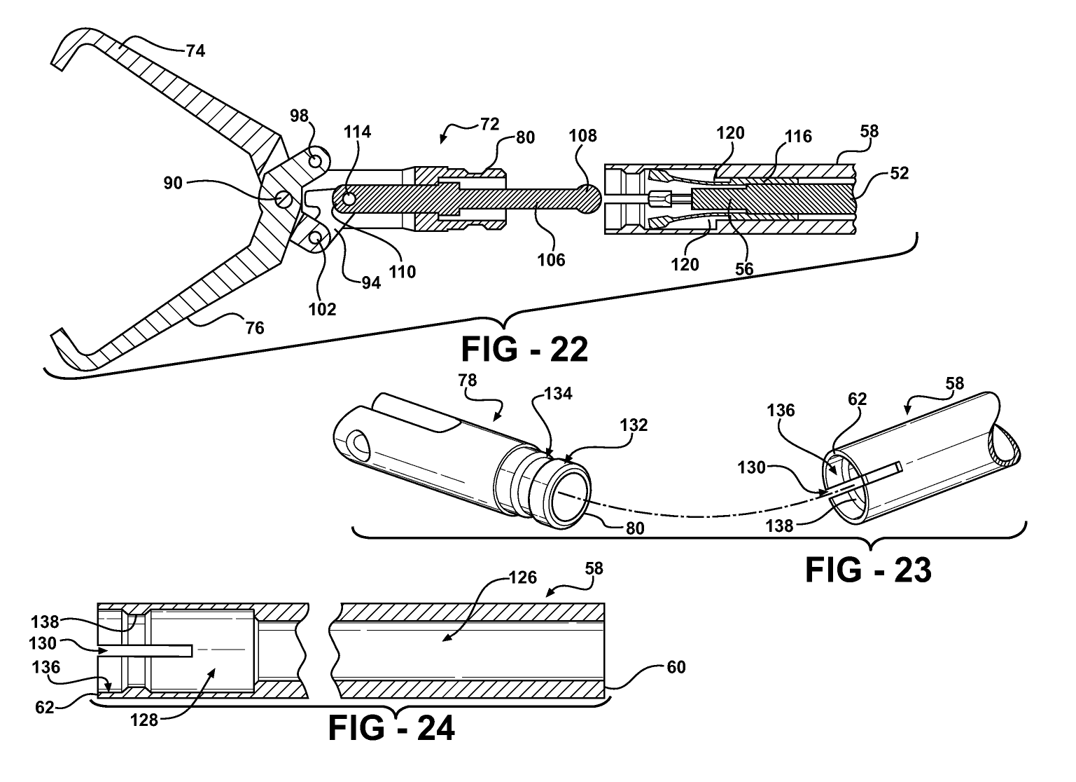

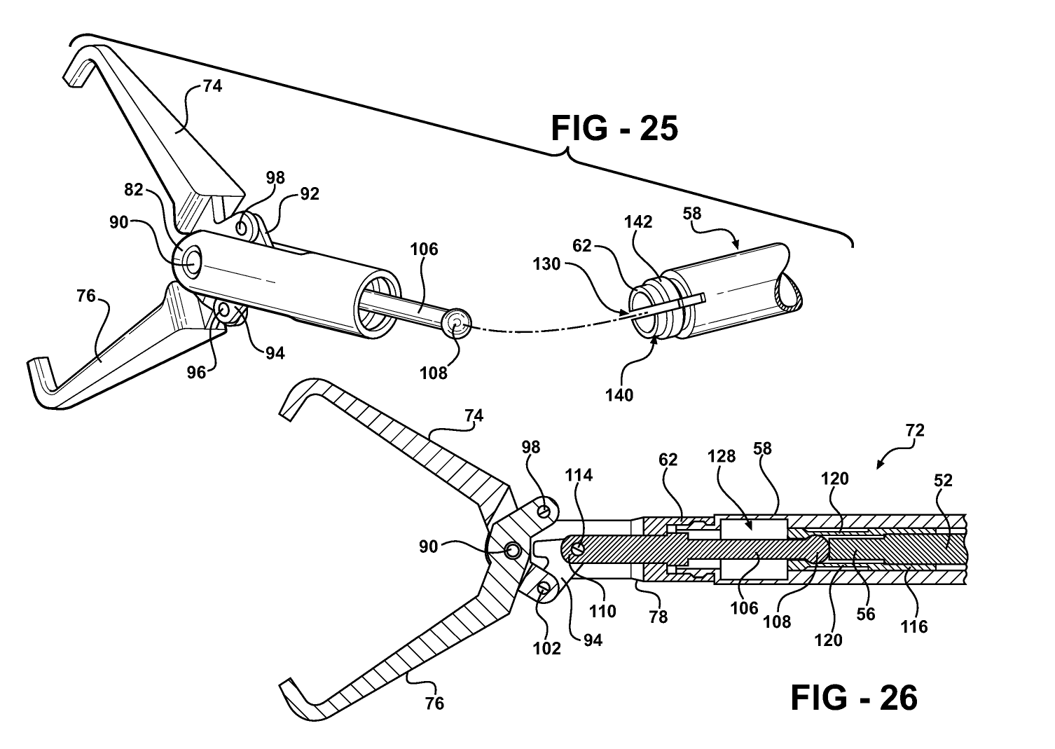

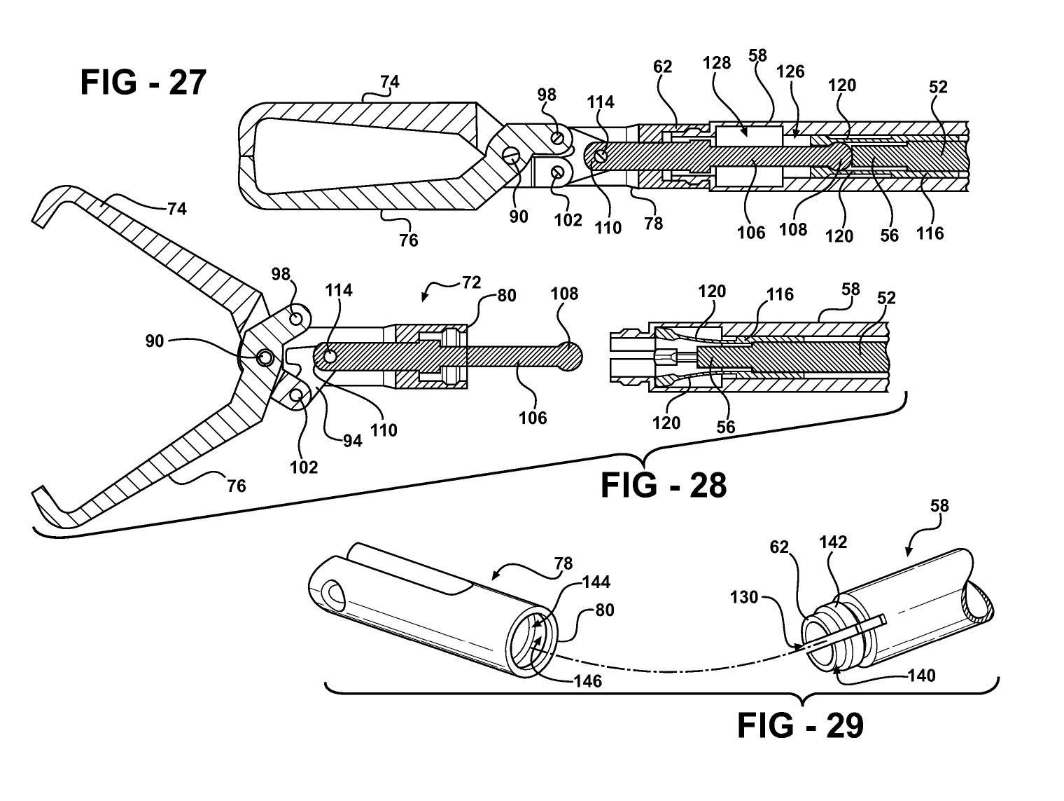

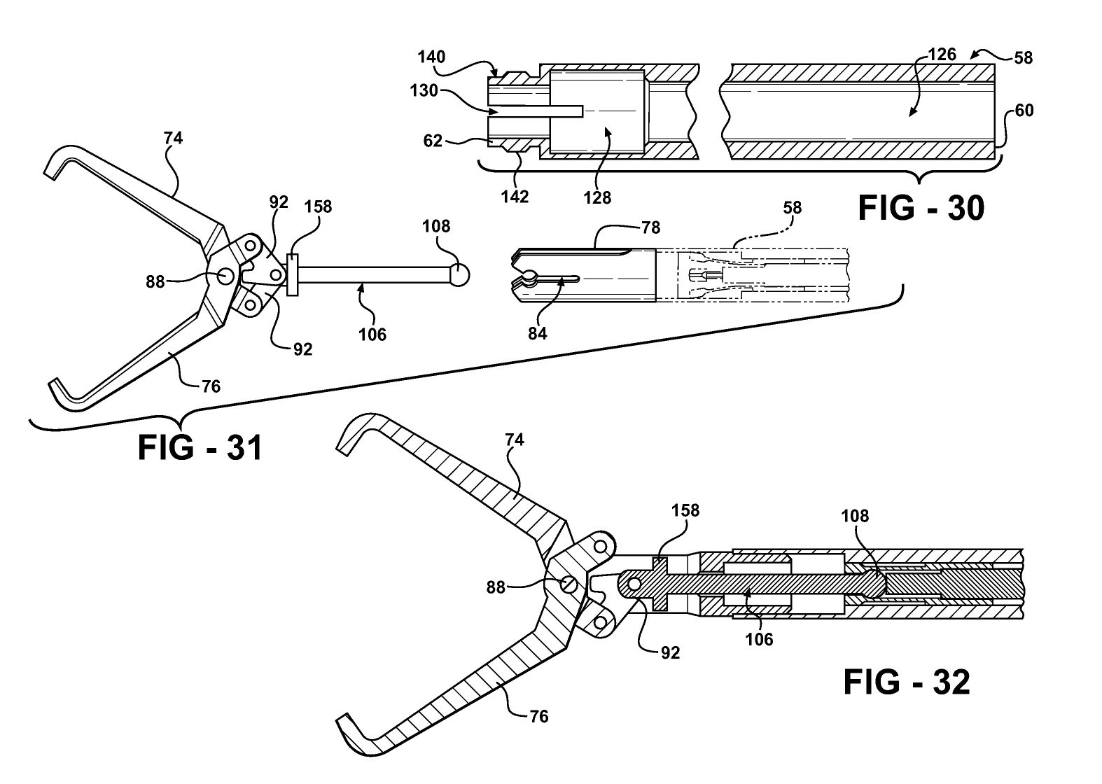

FIGS. 19-24 show a third configuration of the secondary coupling. The third configuration is substantially identical to the first configuration, except that the slit 130 is defined by the housing 58 and projects inward from the distal end 62. A fourth configuration, shown in FIGS. 25-30, is substantially identical to the second configuration, except that the slit 130 is defined by the housing 58 and projects inward form the distal end 62.

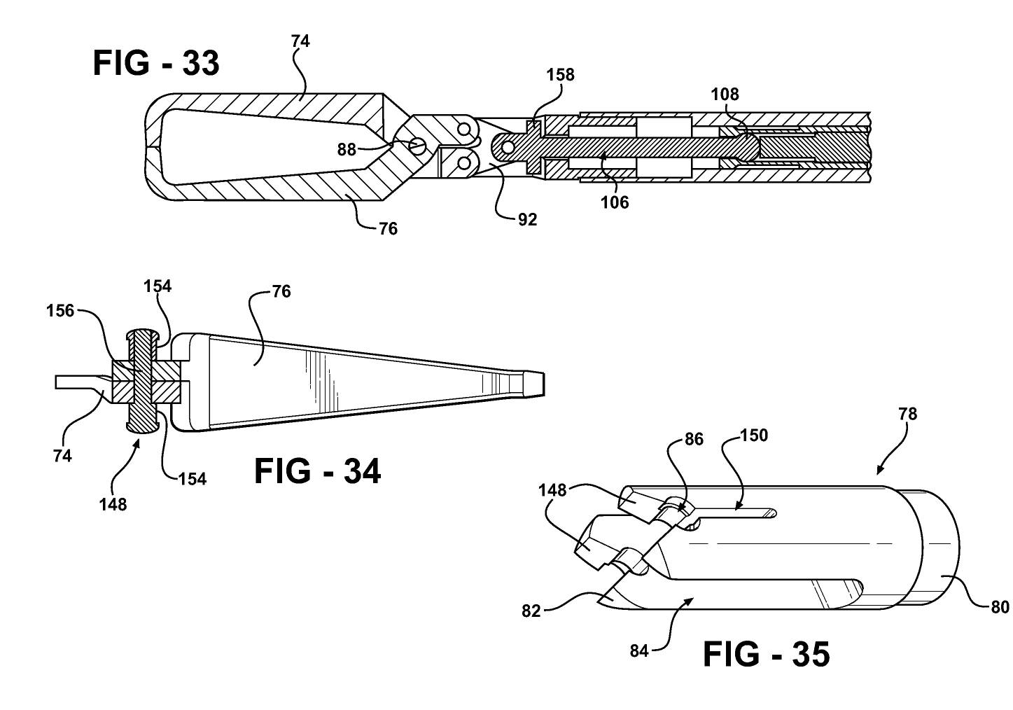

In a second embodiment of the invention, as shown in FIGS. 31-35, the clevis component 78 is affixed to the housing 58. This differs from the first embodiment, where the clevis component 78 is part of the detachable tool assembly 72. Referring to FIG. 33, the proximal end 80 of the clevis component 78 is recessed to mate with the distal end 62 of the housing 58. In the second embodiment, the distal end 82 of the clevis component 78 includes tapered inlets 148. These tapered inlets 148 define a pair of inlet slots 149 extending inward to each clevis hole 86. A slit 150 is also defined projecting inward from each clevis hole 86 to allow the distal end 82 of the clevis component 78 to flex.

In the second embodiment, a pin assembly 152 pivotally connects the jaws 74, 76 via the pivot holes 88. The pin assembly 152 includes a pair of expanded heads 154 interconnected by a pin 156. The pin assembly 152, and specifically the expanded heads 154, extends beyond the jaws 74, 76 on both sides of the jaws 74, 76. To couple the assembly 72 with the housing 58, the pin assembly 152 is slid through the inlet slots 149 and into the clevis holes 86. The expanded heads 154 snap lock with the clevis component 78 in the clevis holes 86. The connection rod 106 carries a perpendicular extension 158. The perpendicular extension 158 mates with the slot 84 of the clevis component 78 which force correct alignment of the assembly 72 and particularly the expandable heads 154 to the clevis holes 86.

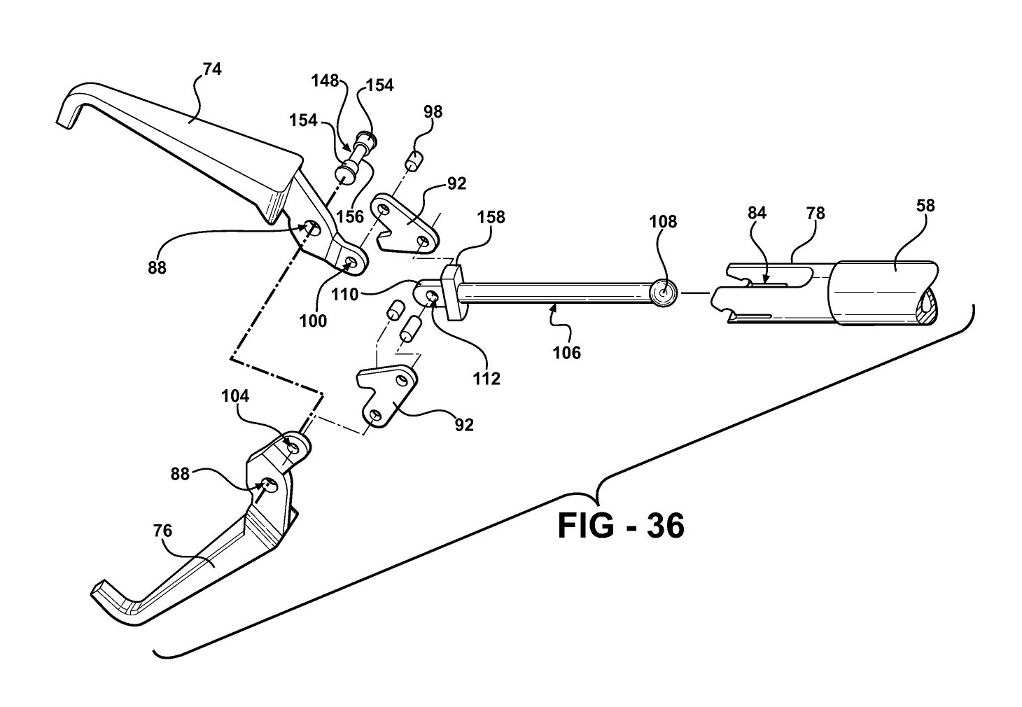

FIGS. 35-41 show a third embodiment of the invention. In the third embodiment the detachable tool assembly 72 is implemented as an electrode 158 for applying electrical current to tissue during a surgical procedure. The electrode 158 is preferably formed as a unitary part, i.e., its components are not fastened together. However, those skilled in the art realize numerous techniques to form the electrode 158.

The electrode 158 includes a proximal end 160 and a distal end 162. In the fourth embodiment, as illustrated, the distal end 162 is ball shaped to provide a round surface for application of electrical energy. However, those skilled in the art realize other shapes for the distal end 162, such as, but not limited to, a needle tip or a scalpel shape.

The electrode 158 forms a void 163 extending inward from the proximal end 160. The electrode 158 includes a connection rod 164 that extends through the void and outward past the proximal end 160. The connection rod 164 includes a ball-shaped end 166. The ball-shaped end mates with the collet 116 such that the electrode 158 may be secured in place or ejected by the fingers 120 of the collet 116.

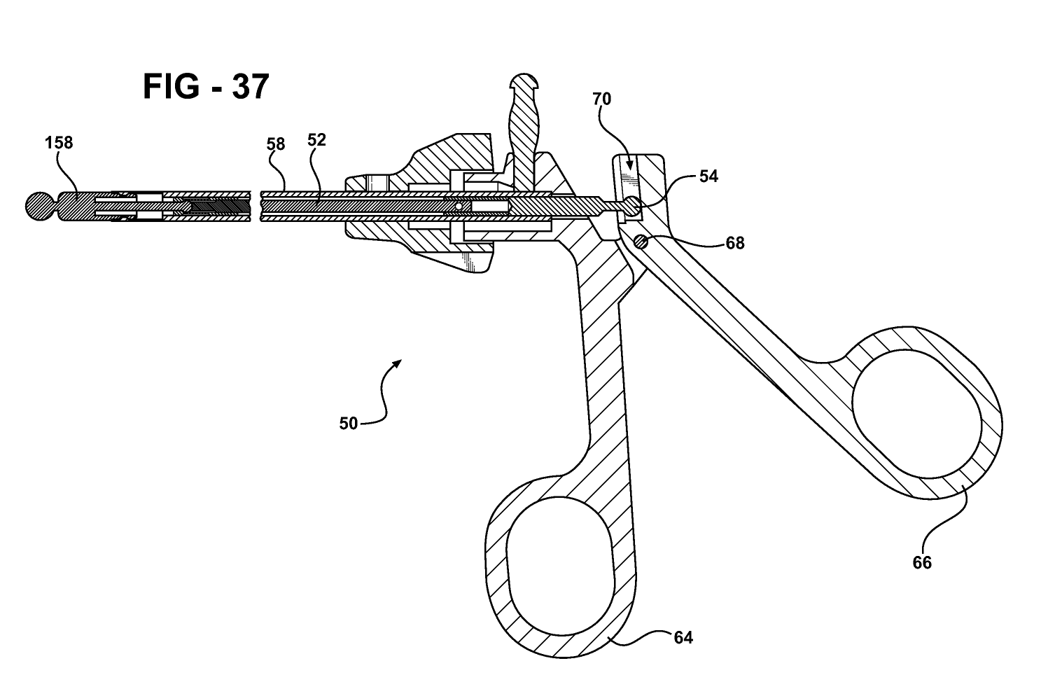

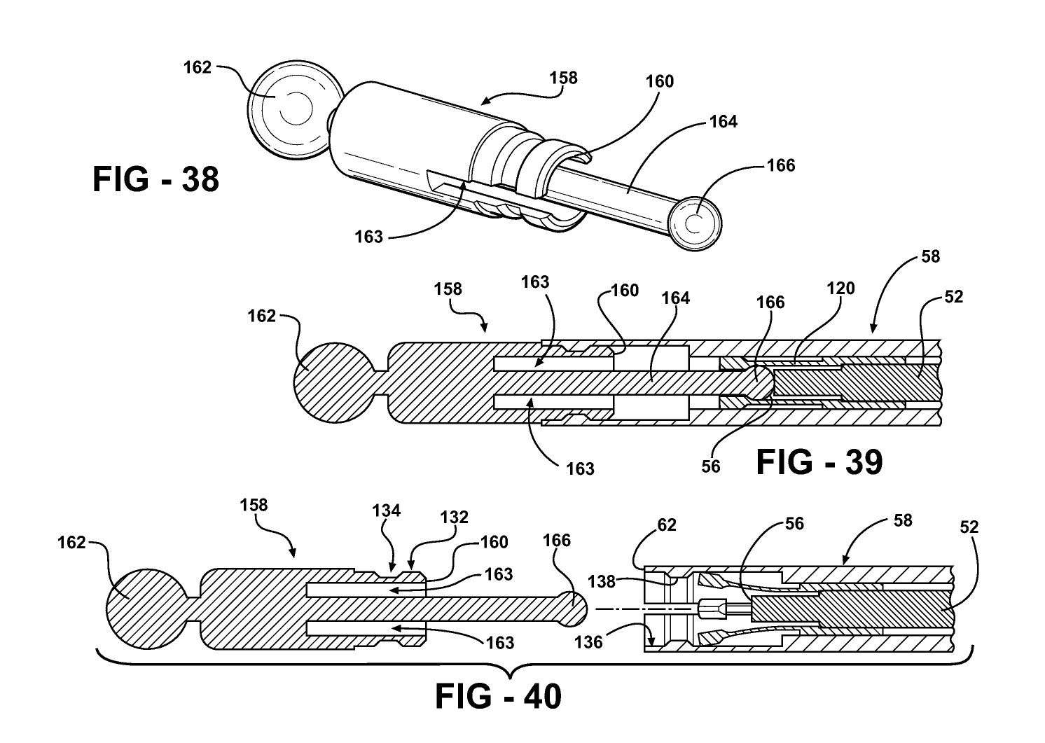

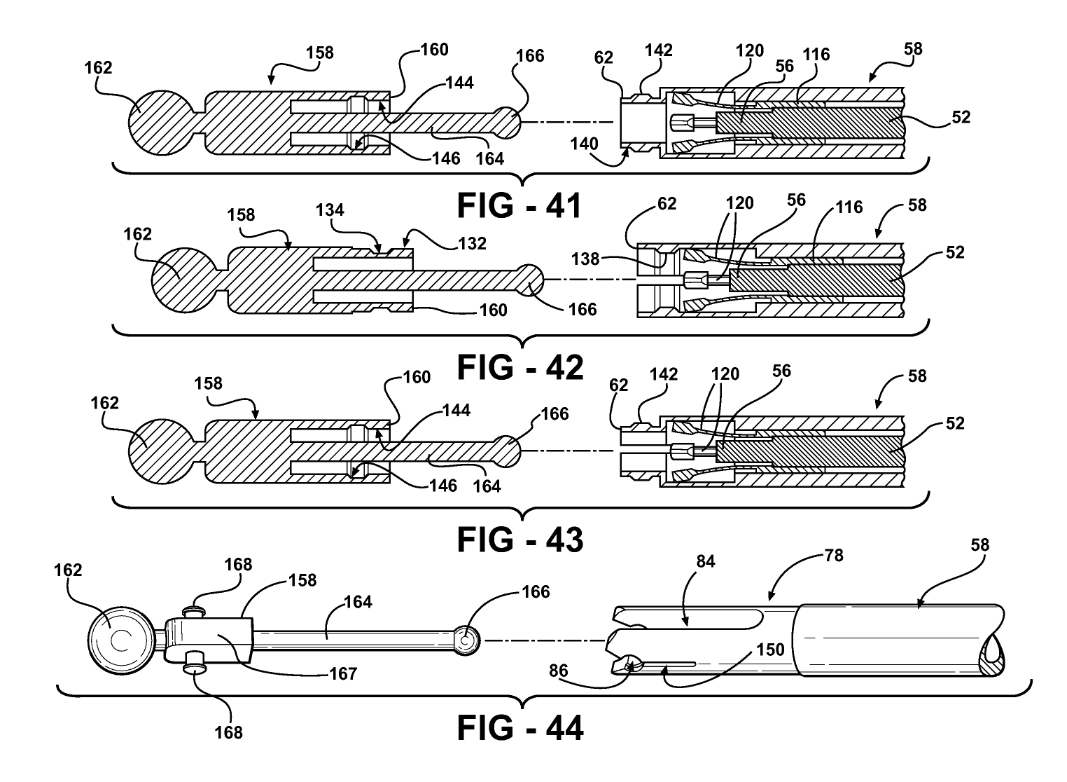

The proximal end 160 of the electrode 158 is detachably connected to the distal end 62 of the housing 58 in a similar fashion to the collet component 78 and housing 58 in the first embodiment. In a first configuration, as shown in FIGS. 38-40, the proximal end 160 of the electrode 158 defines the recessed outer surface 132 with the groove 134. The housing 58 defines the inner surface 136 with the tongue 138 for matting with the groove 134. A second configuration is shown in FIG. 41. In the second configuration, the electrode 158 defines an inner surface 144 with the groove 146 adjacent the proximal end 160. The housing 58 defines the recessed outer surface 140 with the tongue 142 adjacent the distal end 62. A third configuration, shown in FIG. 41, is similar to the first configuration, except the pair of slits 130 are defined in the housing 58 extending inward from the distal end 62. A fourth configuration, shown in FIG. 42, is similar to the second configuration, except a pair of slits 130 are defined in the housing 58 extending inward from the distal end 62.

A fourth embodiment of the surgical instrument 50 is shown in FIG. 44. In this fourth embodiment, the detachable tool assembly is implemented as the electrode 158. The electrode 158 includes a base portion 167. The distal end 162 and the connection rod 162 extend in opposite directions from the base portion 167. A pair of expanded heads 168 extends from the base portion 167 and each head 168 is generally perpendicular to the connection rod 164. The clevis component 78 is attached to the housing 58 and configured as in the second embodiment. Thus, the expanded heads 168 of the electrode 158 snap lock with the clevis holes 86 in the clevis component 78 when attached to the housing 58. In the ejected position, the shaft 52 pushes the electrode 158 away from the housing 58 and forces disengagement of the expanded heads 168 from the clevis holes 86.

The electrode 158 is preferably electrically connected to a power supply (not shown) which provides the electrical current. The electrical current is preferably supplied via the shaft 52 and the connection rod 164. Certain components of the instrument 50, such as the housing 58 and the handles 64, 66, are preferably electrically insulated from the housing 58 and the electrode 158. This electrical insulation may be accomplished by coating the metallic components with an insulating material, such as rubber, or forming the components out of an insulating material, such as plastic.

Other embodiments of the detachable tool assembly 72 may be configured for use with the surgical instrument 50. For instance, the detachable tool assembly 72 may include a pair of blades (not shown) to act as surgical scissors. The detachable tool assembly 72 could also include a single scalpel blade (not shown). Those skilled in the art will realize implementations of the detachable tool assembly.

The present invention has been described herein in an illustrative manner, and it is to be understood that the terminology which has been used is intended to be in the nature of words of description rather than of limitation. Many modifications and variations of the invention are possible in light of the above teachings. The invention may be practiced otherwise than as specifically described within the scope of the appended claims.

1. A surgical instrument comprising:
a shaft having a proximal end and a distal end;
a housing surrounding at least a portion of said shaft;
at least one handle operatively connected to said proximal end of said shaft for longitudinally actuating said shaft within said housing;
a detachable tool assembly including a connection rod having a proximal end and a distal end and at least one tool operatively connected to said connection rod; and
a collet disposed within said housing and detachably coupling said shaft and said connection rod.

2. A surgical instrument as set forth in claim 1 wherein said collet includes a base attached to said distal end of said shaft.

3. A surgical instrument as set forth in claim 2 wherein said collet also includes a plurality of expandable fingers extending from said base such that said fingers capture said proximal end of said connection rod when in a collapsed position and release said proximal end of said connection rod when in an expanded position.

4. A surgical instrument as set forth in claim 1 wherein said housing defines a central opening and a cavity wherein said cavity has a cross-sectional width greater than a cross-sectional width of said central opening.

5. A surgical instrument as set forth in claim 4 wherein said collet is movable within said housing between an ejected position and at least one operating position as said shaft moves longitudinally through said housing wherein said fingers are collapsed within said central opening and capture said ball-shaped proximal end of said connection rod in said at least one operating position and said fingers flare away from one another in said cavity to release said connection rod in said ejected position.

6. A surgical instrument as set forth in claim 3 wherein each of said fingers includes a stem extending from said base and a tip disposed atop said stem.

7. A surgical instrument as set forth in claim 6 wherein said tips of said fingers form a hoop when said collet is in said collapsed position for accommodating said connection rod though said hoop but retaining said proximal end of said connection rod between said tips and said base.

8. A surgical instrument as set forth in claim 1 wherein said at least one tool is further defined as a pair of jaws movable between an open position and a closed position.

9. A surgical instrument as set forth in claim 8 wherein said tool assembly further includes a clevis component defining a hole and detachably coupled to said distal end of said housing.

10. A surgical instrument as set forth in claim 9 wherein said tool assembly further includes a clevis pin pivotally fastening said jaws to said clevis component via said hole.

11. A surgical instrument as set forth in claim 10 wherein said tool assembly includes a pair of links operatively connecting said connection rod to said jaws such that said jaws move between said open and closed positions in response to movement said handle.

12. A surgical instrument as set forth in claim 9 wherein said clevis component defines a groove and said housing includes a tongue for mating with said groove to detachably couple said clevis component with said housing.

13. A surgical instrument as set forth in claim 9 wherein said clevis component includes a tongue and said housing defines a groove for mating with said tongue to detachably couple said clevis component with said housing.

14. A surgical instrument as set forth in claim 1 wherein said at least one tool is further defined as an electrode.

15. A surgical instrument comprising:
a shaft having a proximal end and a distal end;
a housing surrounding at least a portion of said shaft;
at least one handle operatively connected to said proximal end of said shaft for longitudinally actuating said shaft within said housing;
a detachable tool assembly including a connection rod having a proximal end and a distal end and at least one tool operatively connected to said connection rod;
a first coupling formed by a collet disposed within said housing and detachably coupling said shaft and said connection rod;
said detachable tool assembly further including a clevis component; and
a second coupling formed by said clevis component and said housing for detachably coupling said assembly to said housing.

16. A surgical instrument as set forth in claim 15 wherein said collet of said first coupling includes a plurality of expandable fingers such that said fingers capture said proximal end of said connection rod when in a collapsed position and release said proximal end of said connection rod when in an expanded position.

17. A surgical instrument as set forth in claim 15 wherein said clevis component of said second coupling defines a groove and said housing includes a tongue for mating with said groove to detachably couple said clevis component with said housing.

18. A surgical instrument as set forth in claim 15 wherein said clevis component of said second coupling includes a tongue and said housing defines a groove for mating with said tongue to detachably couple said clevis component with said housing.

19. A surgical instrument as set forth in claim 15 wherein said at least one tool is further defined as a pair of jaws movable between an open position and a closed position.

20. A surgical instrument as set forth in claim 19 wherein said clevis component defines a hole and said tool assembly further includes a clevis pin pivotally fastening said jaws to said clevis component via said hole.
